SEQUENCE LISTING (3 sequences)

SEQ ID NO: 1 - DNA, 486 bp, Lactobacillus fermentum
  source 1..486: mol_type = genomic DNA
atgaggaacaatcaaagtaacacgccgctaatttcagtgattattcctgcatataaagtcgaaaaatacttagcgttttgtgttgaatcagttgttgcacaaactttaactggttatgaagtgattattgtcgatgatggctcaccagataatactggagagatcacggatcacttagcgcaacaatatgaagcggttaaggtgattcatcaagaaaacgcaggagttagtaccgcacggaatacggggattgacaacgctcaagggaaatatattacttttattgatggtgatgattttatcgctccgacttttctggagtatatggttaatatggtagagaaaacccattcggatttcggactggctctagattgttttacgaagaatgatgagaaacctttggatcaaactgaagataaagtgtatgctccagaaaaggcggtaagtttgttgctatccccacgtgtaattgttggctgttag

SEQ ID NO: 2 - DNA, 987 bp, Lactobacillus fermentum
  source 1..987: mol_type = genomic DNA
atgaatgatgttccatcaccgtactatgatgaaattatagaacggggttcaaaaatattcattctgccgccaataaataacgtttaccatcattatcaggaatgcaaaaagatattacgagatggggattacgatgttgtttgtgataacaacttgattaaatccatcccaatgatgcttgctgctaagaaatgtggagttcctgtacggattttgcatagtcacaacacgaaattaagcacaatcatcaagaaggaatggattacgaagctcctattgccattattgaaaagggaaattacagattattgtgcatgcggccaactagctggggaagcactatttgggaaagctaagtttacggttattccaaatgtaatctcaccagaaacgaacacctttgataaagtcaagagagataaaatcagaaaagagcttggcgttgacgataaggttgttgtagggactgttggtcggacatctatacaaaaaaacccttatttcgcaattgatgtaattgagaaggtacaccaaagtaatcctagtattgtttattggtggattggtagtggtgaactagatgatcaactgagagcgtacgtagaaaagaaggggttaggtaaggttgtatctttcctaggaagtagggatgatgttcaagatctttaccaggcaatggatgtattctttttaccctcgctttttgaaggtttaccactaactggagttgaagctcaagcaatggggttaccgtcgattttatcagctagcgttacagatagattggtatataccgatctggtaaagtacgtatcgttggatgaacctatcgaagaatgggaaaaagcttttaaaaaagcgatcgaacggattccacaaaggagggcatatacagaagaacttaagcagagcgtttattcagctgaagatgcgggaaagaatatgacaaagatttacgaggatcttctcgcctcaaaattgcggtaa

SEQ ID NO: 3 - DNA, 1086 bp, Lactobacillus fermentum
  source 1..1086: mol_type = genomic DNA
atgattcggatattacaattaccaaacactatttctcgagaaaatggtcgcatgtcagtaattatgagtatatataggcacatagatagaaccaagattcagtttgattttgcggtatctgagtctagtggcgatacttatcttgatgaaattaaaaagcttggaggcaaggtatttgtgattccatctggcgaggtttcctacaaaagtgttgtcaagatggttaatatgctccttaagaaaagagagtattcatttatacattatcacgcaatctcaatttggggagttgctctaaacgttgcacatcggcatggtgtaaagataatcacacacagtcatgcaacatattttagcgatggatttatgaagtcaattcgaaatcgaatcttttctctaaatataaagttatattcagataagttggcagctgtttccccagaagcgggtagaactttatttggaaaacaacaatatatatatataccaaatgtaattaattataaaaaatatactttctcgcgtaataatagagaaaaaattcgtcggcaatataacattgatgatggtgactttgtcgttggtcatgtaggacgtctgtcaaaacaaaaaaaccatcaatttctgatcagagcctttagtctattacatgcatcggcggaaaagtacaaattaatgctcgtgggtagtggaccactcgaaaatgatctgaggacacttgtaagtcaactgaatattgaaaggtcagttatttttgttggtgcaaagcaagatgtaactgcgttttattcagcatttgacttgttctggttaccttccttgtatgagggattgcctacggttggattggaagcgcaggctaacggtctttcaatcattgcaagtgatcgtatttcacctgagctagccattgaaaatgttattttttctccaattaggcataaaagcgatttacaaaaatggtgtcatatcactctggagcgagattggcctcgctctacagatgtcatgcggacgattgaacatagtcggtataattatcaacatgtcttagatcaatggaaaagcctatatgatatgaagtaa

(19) United States
(12) Patent Application Publication
(10) Pub. No.: US 2024/0424014 A1
(43) Pub. Date: Dec. 26, 2024

(54) METHOD FOR TREATING SLEEPING DISORDERS WITH EXOPOLYSACCHARIDES

(71) Applicant: BENED BIOMEDICAL CO., LTD.

(72) Inventors: YING-CHIEH TSAI, TAIPEI CITY (TW); CHIEN-CHEN WU, TAIPEI CITY (TW); CHIN-LIN HUANG, TAIPEI CITY (TW); CHIH-CHIEH HSU, TAIPEI CITY (TW)

(73) Assignee: BENED BIOMEDICAL CO., LTD., TAIPEI CITY (TW)

(21) Appl. No.: 18/686,981

(22) PCT Filed: Aug. 25, 2022
(86) PCT No.: PCT/CN2022/114742

§ 371 Date: Feb. 27, 2024

Related U.S. Application Data
(60) Provisional application No. 63/237,964, filed on Aug. 27, 2021.
(60) Provisional application No. 63/267,326, filed on Jan. 31, 2022.

(51) Int. Cl. A61K 31/715; A61K 35/747; A61P 25/20

(52) U.S. Cl. CPC A61K 31/715; A61K 35/747; A61P 25/20

(57) ABSTRACT

A method for use of exopolysaccharides of a lactic acid bacterium or a pharmaceutical composition containing the exopolysaccharides in the manufacture of a medicament for preventing, ameliorating and/or treating a sleeping disorder in a subject in need thereof. The pharmaceutical composition includes an effective amount of exopolysaccharides (EPSs) of a lactic acid bacterium and optionally an edible carrier or a pharmaceutically acceptable carrier

### FIELD OF THE DISCLOSURE

The present disclosure relates to applications of probiotics. In particular, the present disclosure relates to methods of using lactic acid bacteria.

### BACKGROUND OF THE DISCLOSURE

Probiotics are live microbes that can provide certain beneficial effects to their host. Benefiting from their high commercial value, research in exploring various functions of probiotics has advanced considerably in the past decade. One of the most important milestones is the proposition of “psychobiotics,” which further extend the function of probiotics to behavior and mental health. Besides the discovery of novel applications, many probiotic functions were found to correlate to non-viable cells, which in turn have led to a surge in research on the functional probiotic effectors (Teame T et al., 2020. Paraprobiotics and Postbiotics of Probiotic Lactobacilli, Their Positive Effects on the Host and Action Mechanisms: A Review. Front Nutr 7:570344; Lebeer S wt al., 2018. Identification of probiotic effector molecules: present state and future perspectives. Curr Opin Biotechnol 49:217-223).

In general, functional effectors produced by probiotics can be classified into two major categories: paraprobiotics and postbiotics. Paraprobiotics are inactivated probiotic cells or cellular components of probiotics, which include intracellular proteins, cell wall components, cell surface-associated molecules, and exopolysaccharides (EPSs). Postbiotics are non-cellular components, including secretory molecules and bacterial metabolites. Among the isolate molecules, EPSs are especially interesting due to their diverse chemical properties and their role in host-microbe interactions (Lee I C et al., Strain-Specific Features of Extracellular Polysaccharides and Their Impact on Lactobacillus plantarum-Host Interactions. Appl Eviron Microbiol 82:3959-3970).

However, there is a need to develop new applications for health.

### SUMMARY OF THE INVENTION

The present disclosure provides a method for preventing, ameliorating and/or treating a sleeping disorder in a subject in need thereof, comprising administering to a subject an effective amount of exopolysaccharides of a lactic acid bacterium or a pharmaceutical composition comprising the exopolysaccharides.

The present disclosure also provides use of exopolysaccharides of a lactic acid bacterium or a pharmaceutical composition comprising the exopolysaccharides in the manufacture of a medicament for preventing, ameliorating and/or treating a sleeping disorder in a subject in need thereof.

The present disclosure provides a method for improving sleep quality in a subject in need thereof, comprising administering to a subject an effective amount of exopolysaccharides of a lactic acid bacterium or a pharmaceutical composition comprising the exopolysaccharides.

The present disclosure also provides use of exopolysaccharides of a lactic acid bacterium or a pharmaceutical composition comprising the exopolysaccharides in the manufacture of a medicament for improving sleep quality in a subject in need thereof.

The present disclosure provides a method for preventing, ameliorating and/or treating insomnia in a subject in need thereof, comprising administering to a subject an effective amount of exopolysaccharides of a lactic acid bacterium or a pharmaceutical composition comprising the exopolysaccharides.

The present disclosure also provides use of exopolysaccharides of a lactic acid bacterium or a pharmaceutical composition comprising the exopolysaccharides in the manufacture of a medicament for preventing, ameliorating and/or treating insomnia in a subject in need thereof.

In some embodiments of the disclosure, the lactic acid bacterium described herein is Lactobacillus spp. In one embodiment of the disclosure, the lactic acid bacterium is Lactobacillus fermentum. Examples of Lactobacillus fermentum strains include, but are not limited to, Lactobacillus fermentum Lf2, Lactobacillus fermentum MTCC 25067, or Lactobacillus fermentum PSI50 (which was deposited with Deutsche Sammlung von Mikroorganismen und Zellkulturen under the Budapest Treaty on 6 Jun. 2016 and was given the accession number DSM 32323).

The EPSs as described herein are provided in various forms. In some embodiments of the disclosure, the EPSs are contained in an extract.

Furthermore, in one embodiment, the EPSs are obtained by a process comprising steps of: culturing the lactic acid bacterium to obtain a bacterial culture; and removing bacterial cell bodies from the bacterial culture to obtain a mixture containing the EPSs.

In some embodiments of the disclosure, the process further comprises steps of
precipitating the mixture with ethanol; and
removing ethanol to obtain an extract containing the EPSs.

In some embodiments of the disclosure, the EPSs are obtained by a process comprising steps of:
culturing the lactic acid bacterium to obtain a bacterial culture;
removing bacterial cell bodies from the bacterial culture to obtain a mixture containing the EPSs;
precipitating the mixture with ethanol;
removing ethanol to obtain an extract containing the EPSs; and
obtaining a fraction having a molecular weight ranging from about 9 kDa to about 2000 kDa from the extract.

In one embodiment of the disclosure, the extract comprises a fraction 1 having a molecular weight ranging from about 30 kDa to about 2,000 kDa, and the fraction 1 is separated by size-exclusion chromatography.

In some embodiments of the disclosure, the fraction 1 comprises a major component with a molecular weight of 1,446 kDa, and in one embodiment, the major component accounts for about 74% of the fraction 1.

In some embodiments of the disclosure, the fraction 1 comprises fucose, galactosamine, glucosamine, galactose, glucose, and mannose. In some embodiments, the fraction 1 further comprises other monosaccharides, include but not limited to, myo-inositol and sorbitol. In one embodiment, the fraction 1 comprises about 3.7 mol % to about 5.5 mol % fucose, about 9.0 mol % to about 13.5 mol % galactosamine, about 10.8 mol % to about 16.2 mol % glucosamine, about 27.8 mol % to about 41.7 mol % galactose, about 7.9 mol % to about 11.8 mol % glucose, and about 20.9 mol % to about 31.3 mol % mannose, wherein the total molar percentage of monosaccharide in the fraction 1 is about 100 mol %.

In another embodiment of the disclosure, the extract comprises a fraction 2 having a molecular weight ranging from about 15 kDa to about 750 kDa, and the fraction 2 is divided by size-exclusion chromatography.

In another embodiment of the disclosure, the extract comprises a fraction 3 having a molecular weight ranging from about 9 kDa to about 380 kDa, and the fraction 3 is divided by size-exclusion chromatography.

The administration route of the pharmaceutical composition as described herein may be varied. In one embodiment, the composition is orally administered, and the composition is in a form suitable for oral administration. In another aspect, the composition is in the form of a solid, semi-solid, liquid, or granule of powder.

In some embodiments of the disclosure, the method is for reducing sleep latency.

In some embodiments of the disclosure, the method is for reducing recovery time from sleep.

### BRIEF DESCRIPTION OF THE DRAWINGS

FIG. 1 shows timeline of the pentobarbital induced sleep test. PBS: phosphate buffered saline. This group was used as a normal control. DIPH: diphenhydramine hydrochloride, a well-known antihistamine drug used as a sleep aid. The DIPH was dissolved in PBS (20 mg/mL). This group was used as a positive control. MRS: De Man, Rogosa and Sharpe broth. The unfermented MRS broth was used as a negative control.

FIGS. 2A to 2D show the results of size-exclusion chromatography of PS150 exopolysaccharide containing crude extracts. FIG. 2A shows subfractionation of PS150 exopolysaccharide containing crude extracts. Subfractions are classified into three groups (fractions 1, 2, and 3) according to their retention volume. Carbohydrate content of each subfraction was quantified using a phenol-sulfur method with a glucose reference. FIG. 2B shows molecular weight distribution of fraction 1. FIG. 2C shows molecular weight distribution of fraction 2. FIG. 2D shows molecular weight distribution of fraction 3.

FIGS. 3A to 3D show monosaccharide composition analysis of PS150 exopolysaccharide. FIG. 3A shows monosaccharide standard. FIG. 3B shows monosaccharide composition of fraction 1. FIG. 3C shows monosaccharide composition of fraction 2. FIG. 3D shows monosaccharide composition of fraction 3. The samples were hydrolyzed into monosaccharide and analyzed using high-performance anion exchange chromatography coupled with a pulsed amperometric detector.

FIG. 4A shows the results of sleep latency in Example 2. FIG. 4B shows the results of sleep duration in Example 2. FIG. 4C shows the results of recovery time in Example 2. Data are presented as Mean f SEM. Different superscript letters (a, b) differ significantly at p<0.05 according to one-way ANOVA with a Tukey HSD post-hoc test.

### DETAILED DESCRIPTION OF THE INVENTION

Unless defined otherwise, all scientific or technical terms used herein have the same meaning as those understood by persons of ordinary skill in the art to which the present invention belongs. Any method and material similar or equivalent to those described herein can be understood and used by those of ordinary skill in the art to practice the present invention.

It must be noted that, as used in the specification and the appended claims, the singular forms “a,” “an” and “the” include plural referents unless the context clearly dictates otherwise. Thus, unless otherwise required by context, singular terms shall include the plural, and plural terms shall include the singular.

As used herein, the term “optional” or “optionally” means that the subsequently described event or circumstance may or may not occur, and that the description includes instances where said event or circumstance occurs, and instances where it does not. For example, the phrase “optionally comprising an agent” means that the agent may or may not exist.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein, the term “about” refers to ±20%, preferably ±10%, and even more preferably ±5%.

The term “and/or” is used to refer to both things or either one of the two mentioned.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

The terms “treatment,” “treating,” and “treat” generally refer to obtaining a desired pharmacological and/or physiological effect. The effect may be preventive in terms of completely or partially preventing a disease, disorder, or symptom thereof, and may be therapeutic in terms of a partial or complete cure for a disease, disorder, and/or symptoms attributed thereto. “Treatment” as used herein covers any treatment of a disease in a mammal, preferably a human, and includes (1) suppressing development of a disease, disorder, or symptom thereof in a subject or (2) relieving or ameliorating the disease, disorder, or symptom thereof in a subject.

As used herein, the term “disorder” is used interchangeably with “disease” or “condition.”

As used herein, the term “sleeping disorder” includes conditions recognized by one skilled in the art regarding sleeping disorders; for example, conditions known in the art or conditions that are proposed to be sleeping disorders or discovered to be sleeping disorders. A sleeping disorder also arises in a subject that has other medical disorders, diseases, or injuries, or in a subject being treated with other medications or medical treatments, where the subject, as a result, has difficulty falling asleep and/or remaining asleep, or experiences unrefreshing sleep, e.g., the subject experiences sleep deprivation.

As used herein, “sleep pattern” or “sleep architecture” refers, for a given individual, to the time spent in each of the sleep stages (e.g., REM, N1, N2 and N3, or, alternatively, REM, Stage I, Stage II, Stage II and Stage IV), including the relative proportion of the duration of each stage as compared to the duration of other stages. Parameters of “normal” sleep patterns or sleep architecture for specific populations (e.g., postmenopausal women) are known in the art. See, e.g., Latta et al., 2005, Sleep, 28:1525-1534; Sahlin et al., 2009, Sleep Med., 10:1025-1030.

As used herein, the term “subject” is any animal that can benefit from the administration of a compound or composition as disclosed herein. In some embodiments, the subject is a mammal, for example, a human, a primate, a dog, a cat, a horse, a cow, a pig, a rodent, such as, for example, a rat or mouse. Typically, the mammal is a human.

The term “probiotic” is recognized in the state of the art as a microorganism which, when administered in adequate amounts, confers a health benefit to the host. A probiotic microorganism must fulfil several requirements related to the lack of toxicity, viability, adhesion and beneficial effects. These probiotic features are strain-dependent, even among bacteria of the same species.

The term “effective amount” of an active ingredient as provided herein means a sufficient amount of the ingredient to provide the desired regulation of a desired function. As will be pointed out below, the exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. Thus, it is not possible to specify an exact “effective amount.” However, an appropriate effective amount can be determined by one of ordinary skill in the art using only routine experimentation.

The term “exopolysaccharide” refers to a high-molecular-weight polysaccharide that is secreted by a microorganism.

The term “edible carrier” refers to compounds, materials, compositions, and/or dosage forms which are suitable for use in contact with the tissues of a subject. Each carrier must also be “acceptable” in the sense of being compatible with the other ingredients of the formulation.

The term “pharmaceutically acceptable” as used herein refers to compounds, materials, compositions, and/or dosage forms which are within the scope of sound medical judgment, suitable for use in contact with the tissues of a subject (either a human or non-human animal) without excessive toxicity, irritation, allergic response, or other problem or complication, commensurate with a reasonable benefit/risk ratio. Each carrier, excipient, etc., must also be “acceptable” in the sense of being compatible with the other ingredients of the formulation. Suitable carriers, excipients, etc., can be found in standard pharmaceutical texts.

As used herein, the term “lactic acid bacterium” refers to gram-positive microaerophilic or anaerobic bacteria that ferment sugars while producing acids (including lactic acid as the predominantly produced acid).

The highly diverse chemical composition of various EPSs results in excellent and useful industrial applications, i.e., in emulsifiers, food additives, antioxidants, cryoprotectants, and even nanoparticle stabilizers (Wang J, Salem D R, Sani RK.2019. Extremophilic exopolysaccharides: A review and new perspectives on engineering strategies and applications. Carbohydr Polym 205:8-26; Zhou Y, Cui Y and Qu X. 2019. Exopolysaccharides of lactic acid bacteria: Structure, bioactivity and associations: A review. Carbohydr Polym 207:317-332). Furthermore, EPS also has potential in biomedical applications. One iconic example is the EPS of Lactobacillus rhamnose GG, which can inhibit adipogenesis (Zhang Z et al., 2019. Ability of prebiotic polysaccharides to activate a HIF1α-antimicrobial peptide axis determines liver injury risk in zebrafish. Commun Biol 2:274).

Accordingly, the present disclosure provides a method for preventing, ameliorating and/or treating a sleeping disorder (such as insomnia), improving sleep quality in a subject in need thereof, comprising administering to a subject an effective amount of exopolysaccharides of a lactic acid bacterium or a pharmaceutical composition comprising the exopolysaccharides.

The present disclosure also provides use of exopolysaccharides of a lactic acid bacterium or a pharmaceutical composition comprising the exopolysaccharides in the manufacture of a medicament for preventing, ameliorating and/or treating a sleeping disorder (such as insomnia) and/or improving sleep quality in a subject in need thereof.

The most common lactic acid bacteria are found in “Lactobacillales,” which includes Lactococcus (Lactococcus spp.), Streptococcus (Streptococcus spp.), Lactobacillus (Lactobacillus spp.), Leuconostoc (Leuconostoc spp.), Pediococcus (Pediococcus spp.), and Enterococcus (Enterococcus spp.). In addition, lactic acid producing bacteria belonging to the group of the strictly anaerobic bacteria Bifidobacterium (i.e., Bifidobacterium spp.) are generally included in the group of lactic acid bacteria.

As the most well-studied probiotic genus, Lactobacillus spp. are the main subject for the isolation of bioactive EPS. As a common species in various fermented foods, L. fermentum is known for its slimy texture and EPS-producing capability, which makes it an ideal source for novel EPS discovery. EPS producing strains, such as L. fermentum Lf2 and L. fermentum MTCC 25067, were extensively studied for the chemical structure, genetics, rheological properties, and production of EPS. More importantly, there is increasing evidence (yet preliminary) that connects the probiotic function of L. fermentum strains to EPS. Particularly, the lactic acid bacterium is Lactobacillus fermentum PS150. Lactobacillus fermentum PS150 is disclosed in WO2018129722A1, and has been deposited with Deutsche Sammlung von Mikroorganismen and Zellkulturen under the Budapest Treaty with the accession number DSM 32323.

Lactobacillus fermentum PS150 (PS150) has any of the nucleic acid sequences as shown in SEQ ID NOs: 1 to 3. The sequences of SEQ ID NOs; 1 to 3 are listed below.

| No | Gene Description | Gene Sequence |
|---|---|---|
| 1 | Putative | atgaggaacaatcaaagtaacacgccgctaatttcagtgattattcctgcatataaagtcgaa |
| | glycosyltransferase | aaatacttagcgttttgtgttgaatcagttgttgcacaaactttaactggttatgaagtgattattg |
| | EpsJ | tcgatgatggctcaccagataatactggagagatcacggatcacttagcgcaacaatatga |
| | | agcggttaaggtgattcatcaagaaaacgcaggagttagtaccgcacggaatacggggat |
| | | tgacaacgctcaagggaaatatattacttttattgatggtgatgattttatcgctccgacttttct |
| | | ggagtatatggttaatatggtagagaaaacccattcggatttcggactggctctagattgtttt |
| | | acgaagaatgatgagaaacctttggatcaaactgaagataaagtgtatgctccagaaaagg |
| | | cggtaagtttgttgctatccccacgtgtaattgttggctgttag (SEQ ID NO: 1) |
| 2. | Putative | atgaatgatgttccatcaccgtactatgatgaaattatagaacggggttcaaaaatattcattct |
| | glycosyltransferase | gccgccaataaataacgtttaccatcattatcaggaatgcaaaaagatattacgagatgggg |
| | EpsF | attacgatgttgtttgtgataacaacttgattaaatccatcccaatgatgcttgctgctaagaaa |
| | | tgtggagttcctgtacggattttgcatagtcacaacacgaaattaagcacaatcatcaagaag |
| | | gaatggattacgaagctcctattgccattattgaaaagggaaattacagattattgtgcatgc |
| | | ggccaactagctggggaagcactatttgggaaagctaagtttacggttattccaaatgtaatc |
| | | tcaccagaaacgaacacctttgataaagtcaagagagataaaatcagaaaagagcttggc |
| | | gttgacgataaggttgttgtagggactgttggtcggacatctatacaaaaaaacccttatttcg |
| | | caattgatgtaattgagaaggtacaccaaagtaatcctagtattgtttattggtggattggtagt |
| | | ggtgaactagatgatcaactgagagcgtacgtagaaaagaaggggttaggtaaggttgtat |
| | | ctttcctaggaagtagggatgatgttcaagatctttaccaggcaatggatgtattctttttaccc |
| | | tcgctttttgaaggtttaccactaactggagttgaagctcaagcaatggggttaccgtcgattt |
| | | tatcagctagcgttacagatagattggtatataccgatctggtaaagtacgtatcgttggatga |
| | | acctatcgaagaatgggaaaaagcttttaaaaaagcgatcgaacggattccacaaaggag |
| | | ggcatatacagaagaacttaagcagagcgtttattcagctgaagatgcgggaaagaatatg |
| | | acaaagatttacgaggatcttctcgcctcaaaattgcggtaa (SEQ ID NO: 2) |
| 3 | Putative | atgattcggatattacaattaccaaacactatttctcgagaaaatggtcgcatgtcagtaattat |
| | glycosyltransferase | gagtatatataggcacatagatagaaccaagattcagtttgattttgcggtatctgagtctagt |
| | EpsF | ggcgatacttatcttgatgaaattaaaaagcttggaggcaaggtatttgtgattccatctggcg |
| | | aggtttcctacaaaagtgttgtcaagatggttaatatgctccttaagaaaagagagtattcattt |
| | | atacattatcacgcaatctcaatttggggagttgctctaaacgttgcacatcggcatggtgtaa |
| | | agataatcacacacagtcatgcaacatattttagcgatggatttatgaagtcaattcgaaatcg |
| | | aatcttttctctaaatataaagttatattcagataagitggcagctgtttccccagaagcgggta |
| | | gaactttatttggaaaacaacaatatatatatataccaaatgtaattaattataaaaaatatacttt |
| | | ctcgcgtaataatagagaaaaaattcgtcggcaatataacattgatgatggtgactttgtcgtt |
| | | ggtcatgtaggacgtctgtcaaaacaaaaaaaccatcaatttctgatcagagcctttagtctat |
| | | tacatgcatcggcggaaaagtacaaattaatgctcgtgggtagtggaccactcgaaaatga |
| | | tctgaggacacttgtaagtcaactgaatattgaaaggtcagttatttttgttggtgcaaagcaa |
| | | gatgtaactgcgttttattcagcatttgacttgttctggttaccttccttgtatgagggattgccta |
| | | cggttggattggaagcgcaggctaacggtctttcaatcattgcaagtgatcgtatttcacctg |
| | | agctagccattgaaaatgttattttttctccaattaggcataaaagcgatttacaaaaatggtgt |
| | | catatcactctggagcgagattggcctcgctctacagatgtcatgcggacgattgaacatag |
| | | tcggtataattatcaacatgtcttagatcaatggaaaagcctatatgatatgaagtaa (SEQ |
| | | ID NO: 3) |

Typically, exopolysaccharides comprise a polymer of monosaccharides. Some exopolysaccharides, however, also comprise non-carbohydrate substituents (such as acetate, pyruvate, succinate, and phosphate). Depending on the monosaccharide composition, EPS can be classified into homopolysaccharides and heteropolysaccharides. Homopolysaccharides are composed of repeating units constructed by a single type of monosaccharide. However, repeating units of heteropolysaccharides comprise multiple types of monosaccharides. Besides monosaccharide composition, factors including the number of repeating units, types of glycosidic bonds, branches, sulfation, and phosphorylation could vary in EPS produced by a different organism.

As described herein, intervention of EPSs can reduce sleep latency, although sleep duration is not affected after the treatment. On the other hand, the EPSs treatment leads to significantly reduced recovery time. These results revealed that the EPSs are sufficient to produce a hypnotic effect.

The EPSs as described herein may be provided in a purified, semi-purified or unpurified form. In some embodiments, the EPSs as described herein are semi-purified and contained in an extract. The extract may be prepared by a process comprising steps of:
culturing the lactic acid bacterium to obtain a bacterial culture; and
removing bacterial cell bodies from the bacterial culture to obtain a mixture.

Additionally, the process for preparing the extract further comprises steps of
precipitating the mixture with ethanol; and
removing ethanol to obtain an extract containing the EPSs.

Still moreover, the process for preparing the extract further comprises steps of obtaining a fraction having a molecular weight ranging from about 9 kDa to about 2,000 kDa from the extract.

Furthermore, the extract containing EPSs as described herein may be further divided into several fractions. The manner of dividing may be size-exclusion chromatography. The extract comprises some fractions. One of the fractions is fraction 1 having a molecular weight ranging from about 30 kDa to about 2,000 kDa, and the fraction 1 is divided by size-exclusion chromatography; one of the fractions is fraction 2 having a molecular weight ranging from about 15 kDa to about 750 kDa, and the fraction 2 is divided by size-exclusion chromatography; one of the fractions is fraction 3 having a molecular weight ranging from about 9 kDa to about 380 kDa, and the fraction 3 is divided by size-exclusion chromatography.

The term “size-exclusion chromatography” means a method for separating molecules using porous chromatographic material. Size-exclusion chromatography can consist of one or more distinct types of porous chromatographic material used in a single step, or one or more distinct types of porous chromatographic material used in multiple separate steps.

In some embodiments of the disclosure, the fraction 1 comprises a major component with a molecular weight of about 1,446 kDa. Further analyzing contents in the fraction 1 using a high-performance anion-exchange column chromatography system (HPAEC) with a pulsed amperometric detector (PAD) using a gold working electrode and an anion-exchange column (Carbopac™ PA-10, 4.6×250 mm) (Dionex™ BioLC), the fraction 1 comprises about 3.7 mol % to about 5.5 mol % fucose, about 9.0 mol % to about 13.5 mol % galactosamine, about 10.8 mol % to about 16.2 mol % glucosamine, about 27.8 mol % to about 41.7 mol % galactose, about 7.9 mol % to about 11.8 mol % glucose, and about 20.9 mol % to about 31.3 mol % mannose, wherein the total molar percentage of monosaccharide in the fraction 1 is 100 mol %. Quantification analysis shows that galactose and mannose are most abundant in the fraction 1.

A subject in need of improved sleep architecture or sleep quality may exhibit sleep patterns that deviate from “normal” sleep patterns in one or more parameters, may suffer from or be at risk of perturbed sleep patterns, or may be diagnosed with a sleep disorder. In specific embodiments, the subject has a problem falling asleep.

In determining whether a drug or treatment protocol achieves improved sleep architecture or sleep quality, a number of different parameters may be considered. For example, one or more of a decrease in wake time, increase in slow wave (SW) and/or rapid eye movement (REM) sleep, increase in sleep maintenance, increase in sleep efficiency, decrease in sleep latency and/or normalization of distribution of SW and REM stages during sleep, may be considered to be improvements in sleep quality or sleep architecture. The methods by which one can determine improvement in sleep quality are known in the art and illustrated in the example. A drug or treatment protocol offering improvements in any one or more of these parameters to a subject would be considered to have improved the sleep quality or sleep architecture of that subject.

The compositions of the disclosure can be in any form suitable for administration, in particular, oral administration. This includes, for instance, solids, semi-solids, liquids, and powders.

Examples of the compositions of the disclosure are nutritional compositions, including food products, and in particular, dairy products.

The composition can be, for example, a capsule, tablet, drink, powder or dairy product. Preferably, the present composition is a nutraceutical or a pharmaceutical product, a nutritional supplement or medical food.

Nutritional compositions of the disclosure also include food supplements, and functional food. A “food supplement” designates a product made from compounds usually used in foodstuffs, but which is in the form of tablets, powder, capsules, potion or any other form not usually associated with aliments, and which has beneficial effects on one's health. A “functional food” is an aliment which also has beneficial effects on one's health. In particular, food supplements and functional food can have a physiological effect—protective or curative—against a disease.

If the composition according to the disclosure is a dietary supplement, it can be administered as such, can be mixed with a suitable drinkable liquid, such as water, yoghurt, milk or fruit juice, or can be mixed with solid or liquid food. In this context, the dietary supplement can be in the form of tablets, pills, capsules, lozenges, granules, powders, suspensions, sachets, pastilles, sweets, bars, syrups and corresponding administration forms, usually in the form of a unit dose. Preferably, the dietary supplement comprising the composition of the disclosure is administered in the form of tablets, lozenges, capsules or powders, manufactured in conventional processes of preparing dietary supplements.

The compositions described herein can be pharmaceutically acceptable compositions, which may include one or more pharmaceutically acceptable carriers, excipients, binders, diluents or the like. The instant compositions can be formulated for various routes of administration, for example, by oral administration. They also may be provided in combination with delivery vehicles such as those in some encapsulating technology.

For oral administration, powders, suspensions, granules, tablets, pills, capsules, gelcaps, and caplets are acceptable as solid dosage forms. These can be prepared by, for example, mixing one or more compounds disclosed herein with at least one additive such as starch or another additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as parabens or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and compositions may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

It is to be understood that if any prior art publication is referred to herein, such reference does not constitute an admission that the publication forms a part of the common general knowledge in the art.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

### EXAMPLES

### Purification of Exopolysaccharide Containing Crude Extract

Bacterial strain Lactobacillus fermentum PSI50 was cultured in De Man, Rogosa and Sharpe broth (MRS; Criterion, Hardy diagnostics, Santa Maria, CA, USA) anaerobically at 37° C. for 18 h. The overnight culture was heated at 80° C. for 1 h and the bacterial cell bodies were removed using centrifugation at 7,000×g for 30 min. The polysaccharide component was then precipitated using 3× volume of anhydrous ethanol at 4° C. for 24 h. The precipitants were then collected at 7,000×g for 30 min and washed by 70% ethanol. The residual ethanol was removed by evaporation and then the EPS containing extract was dissolved in ddH₂O and stored in −30° C. until use.

### Size-Exclusion Column Chromatography

Briefly, 40 mg lyophilized EPS was dissolved in 3 mL buffer containing 150 mM NaCl and 10 mM NaH₂PO₄ at pH 6.8 and applied on a Fractogel™ BioSec column (102×1.5 cm) (Merck, N.J., U.S.A.). Then, 2.8 mL/tube was collected and assayed for hexose by the phenol sulfuric acid method (DuBois DuBois, Al., Gilles, K. A., Hamilton, J. K., Rebers, PA., and, Smith, F Colorimetric method for determination of sugars and related substances, Anal Chem 28: 350-356et al., 1956). Spectrophotometric analysis was performed at a 488 nm wavelength.

### Estimation of Exopolysaccharides Molecular Weight

The saccharides containing tubes collected by size-exclusion column were divided into three fractions. Each fraction was further separated using gel-permeation chromatography coupled with a refractive index detector to estimate the molecular weight. A calibration curve was constructed using authentic standards (Dextran series, Sigma-Aldrich Co.) with molecular weights of 670.0×10³, 69.8×10³, 40.0×10³, 10.5×10³ and 0.18×10³ Da.

### Monosaccharide Composition Analysis

The monosaccharide composition of EPS was analyzed using a high-performance anion-exchange column chromatography system (HPAEC) with a pulsed amperometric detector (PAD) using a gold working electrode and an anion-exchange column (Carbopac™ PA-10, 4.6×250 mm) (Dionex™ BioLC). The EPS (1 mg) was acid hydrolyzed with 1.95 N trifluoroacetic acid at 80° C. for 6 h, evaporated to remove the residual TFA, and dissolved in Milli-Q water. The aqueous phase was filtered through a 0.22-μm filter before HPAEC analysis. Isocratic NaOH (18 mM) at ambient temperature was employed as the eluent. The flow rate was 1.0 mL/min. Ten monosaccharide standards, including myo-inositol, sorbitol, mannitol, fucose, galactosamine, glucosamine, galactose, glucose, mannose, and fructose, were purchased from Sigma-Aldrich Co. (St. Louis, MO, USA). Data were collected and integrated on a PRIME DAK system (HPLC Technology, Ltd. UK).

### Animals

Male C57BL/6J mice (6 weeks old) were purchased from the National Laboratory Animal Center (Taipei, Taiwan). The mice were housed in the Laboratory Animal Center of National Yang-Ming University. The room was kept at a constant temperature (22±1° C.) and humidity (55-65%) with a 12 hr light/dark cycle. The mice were fed ad libitum with standard chow diet and sterilized water.

### Pentobarbital Induced Sleep Test

For the functional validation of EPS containing crude extract, the C57BU/6J mice were orally administered with PBS, MRS broth, and EPS (0.4 mg/day) for 13 days. On day 14, the mice were administered with PBS, DIPH (20 mg/kg), MRS broth, or EPS 30 min before the injection of pentobarbital (50 mg/kg). After the injection, the mice were tested for righting reflex by flipping upside down. The time between pentobarbital injection and the loss of righting reflex was defined as sleep latency. Time required for restoration of righting reflex was defined as sleep duration, whereas time spent between righting action and voluntary movement was defined as recovery time.

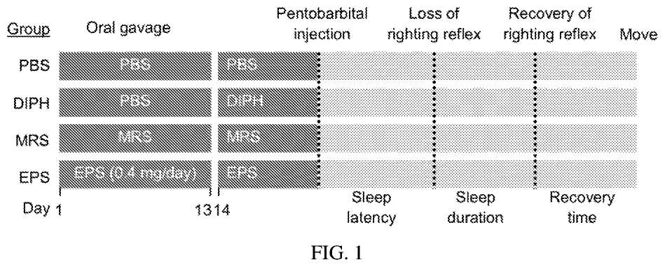

The timeline of the pentobarbital induced sleep test is shown in FIG. 1.

### Example 1 Subfractionation and Characterization of PS150 EPS

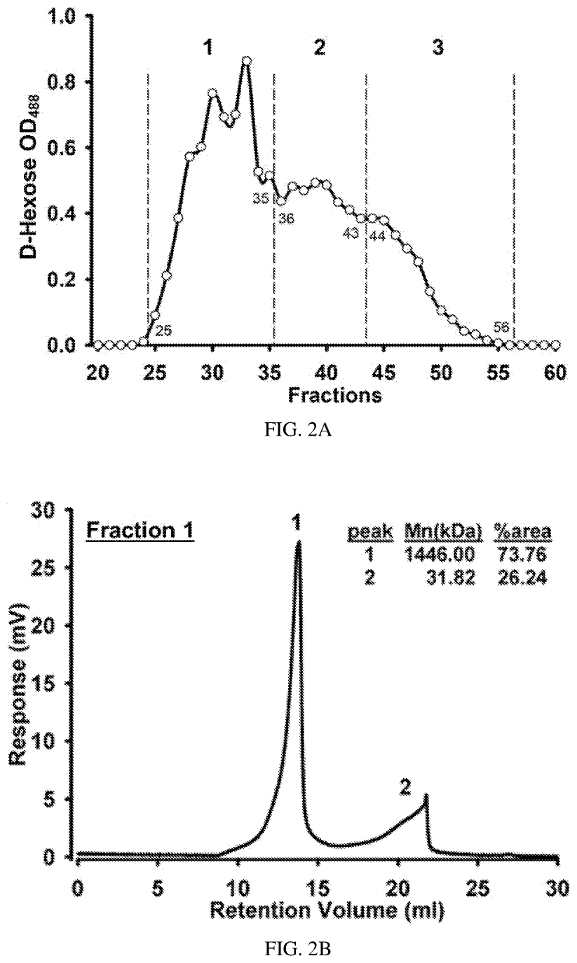

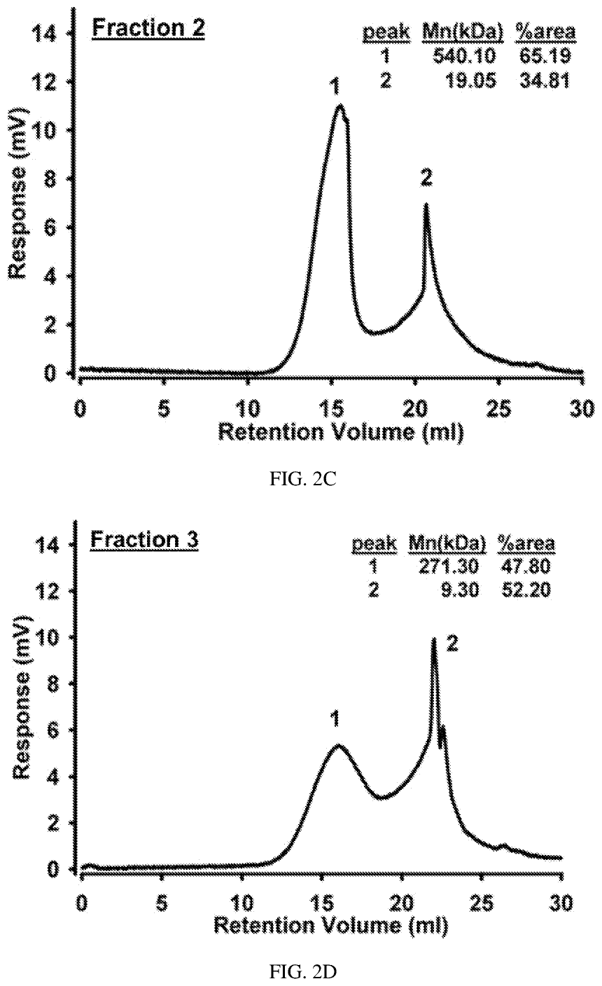

To investigate the composition of EPS, we performed size-exclusion column chromatography for fractionation (FIG. 2A). EPS was separated into three subfractions, namely fractions 1, 2, and 3. Next, we analyzed the homogeneity of the three fractions using a gel-permeation chromatography system. Interestingly, fraction 1 was composed of a major component (74%) with a molecular weight of 1,446 kDa (FIG. 2B). In contrast to the high purity of fraction 1, the chromatograms of fraction 2 and fraction 3 revealed miscellaneous molecular weight distribution and higher background noise (FIGS. 2C and 2D). Accordingly, we speculate that both fraction 2 and fraction 3 are mixtures of various molecules, whereas fraction 1 is purified EPS.

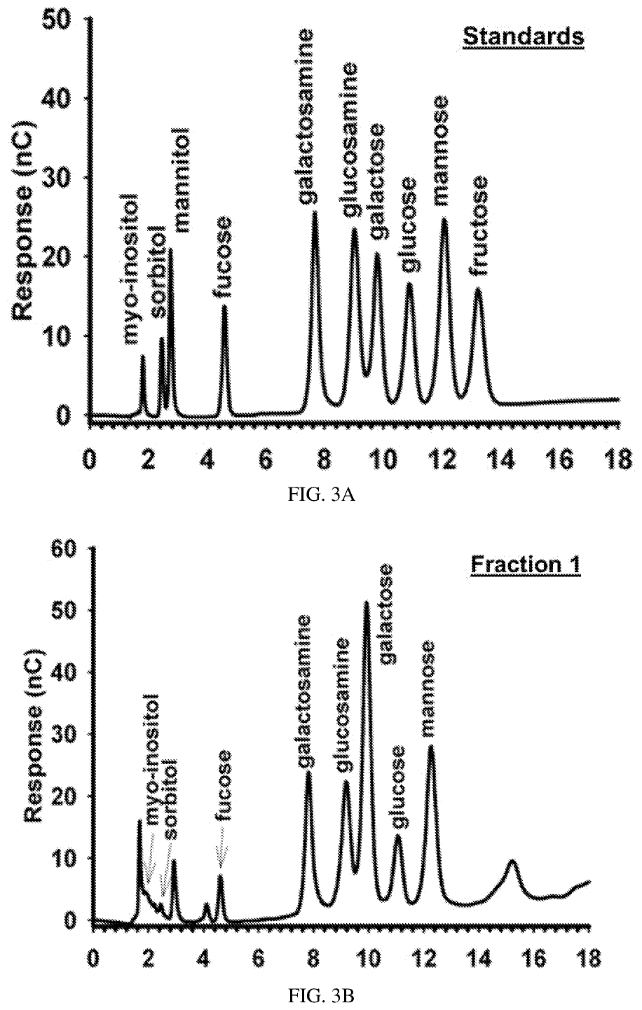

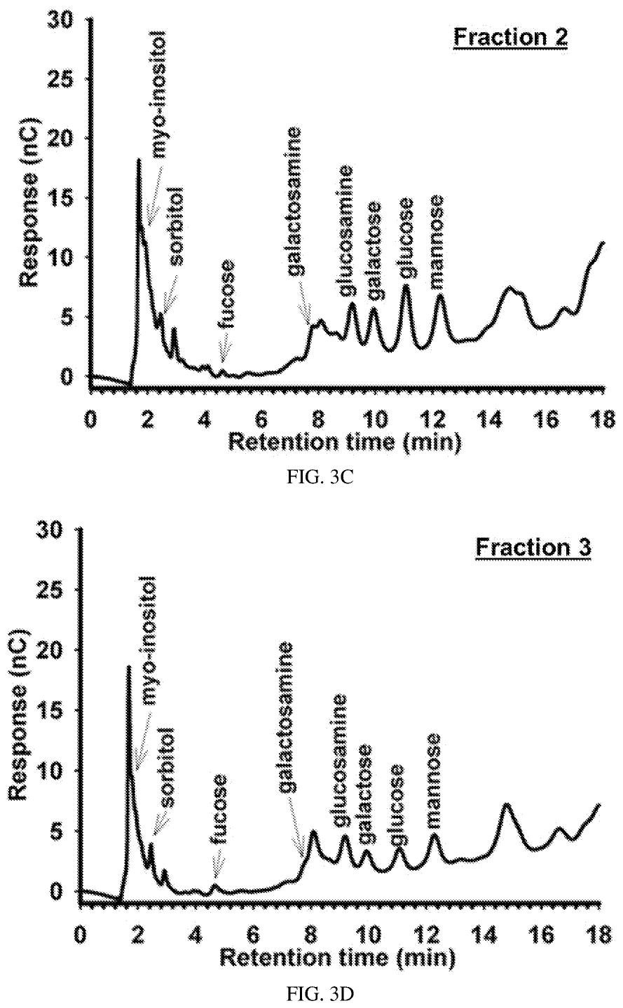

Next, a monosaccharide composition analysis was performed to further characterize the chemical properties of the three fractions (FIGS. 3A to 3D). The results revealed that fraction 1 is a polysaccharide mainly composed of six different monosaccharides: fucose, galactosamine, glucosamine, galactose, glucose, and mannose. Quantification analysis showed that galactose and mannose were the most abundant components of fraction 1 (Table 1). The background noise of fraction 2 and fraction 3 persisted in our monosaccharide analysis (FIGS. 3C and 3D) and the quantification analysis of monosaccharide composition were also shown in Table 1. Collectively, we concluded that the major component of EPS is a heteropolysaccharide composed of six monosaccharides, and such EPS molecule could be the active component mediating the hypnotic effect of PS150.

TABLE 1
Monosaccharide composition of purified fractions.

| | Neutral sugars (μmol/g polysaccharide) | | |
|---|---|---|---|
| Monosaccharide | Fraction 1 | Fraction 2 | Fraction 3 |
| myo-inositol | 1.28 ± 0.01 | 6.13 ± 0.04 | 10.48 ± 0.15 |
| sorbitol | 3.50 ± 0.08 | 5.65 ± 0.06 | 7.87 ± 0.01 |
| fucose | 49.94 ± 1.07 | 3.27 ± 0.02 | 7.96 ± 0.06 |
| galactosamine | 122.26 ± 0.41 | 12.24 ± 0.03 | 5.68 ± 0.42 |
| glucosamine | 146.39 ± 0.50 | 31.47 ± 0.42 | 17.35 ± 0.21 |
| galactose | 377.08 ± 2.17 | 31.45 ± 0.36 | 12.80 ± 0.21 |
| glucose | 106.77 ± 0.41 | 52.14 ± 0.04 | 21.12 ± 0.02 |
| mannose | 283.11 ± 0.51 | 52.95 ± 0.38 | 38.11 ± 0.27 |

### Example 2 the EPS Containing Crude Extract is Sufficient for the Hypnotic Function

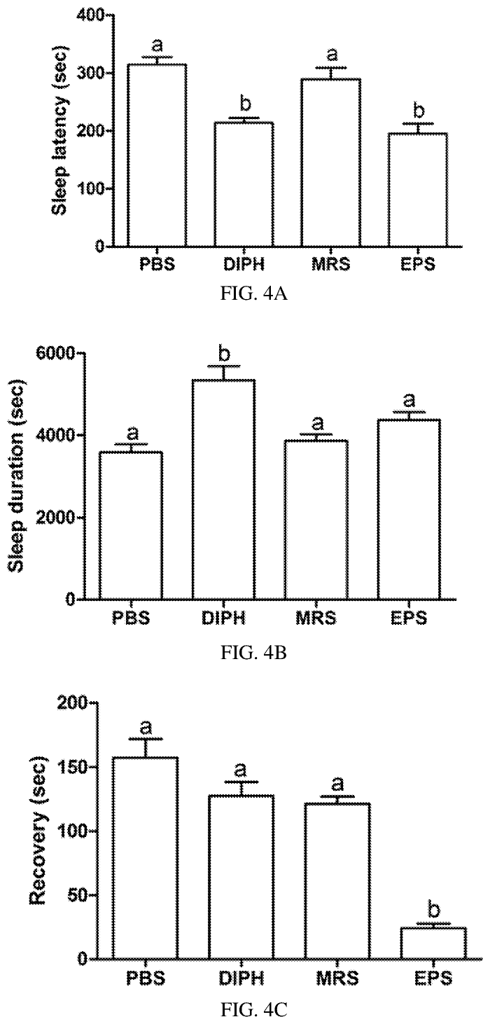

To testify our hypothesis that EPS of PS150 is the active effector for the hypnotic function, we precipitated EPS from the spent medium of PS150. The final yield of carbohydrate content in the crude extract was around 0.4 mg per ml of liquid culture. The hypnotic activity of the crude extract was subsequently tested in a pentobarbital injected mice model as described in Pentobarbital induced sleep test. The result shows that intervention of EPS containing extract from the spent medium of PS150 can reduce sleep latency (FIG. 4A), although sleep duration is not affected after the treatment (FIG. 4B). On the other hand, the EPSs treatment leads to significantly reduced recovery time (FIG. 4C).

While the present disclosure has been described in conjunction with the specific embodiments set forth above, many alternatives thereto and modifications and variations thereof will be apparent to those of ordinary skill in the art. All such alternatives, modifications and variations are regarded as falling within the scope of the present disclosure.

1-17. (canceled)

18. A method for preventing, ameliorating and/or treating a sleeping disorder and/or improving sleep quality in a subject in need thereof comprising administering exopolysaccharides of a lactic acid bacterium or a pharmaceutical composition comprising the exopolysaccharides to the subject.

19. The method of claim 18, wherein the sleeping disorder is insomnia.

20. The method of claim 18, wherein the lactic acid bacterium is Lactobacillus spp.

21. The method of claim 18, wherein the lactic acid bacterium is Lactobacillus fermentum.

22. The method of claim 18, wherein the lactic acid bacterium is Lactobacillus fermentum Lf2, Lactobacillus fermentum MTCC 25067, or Lactobacillus fermentum PS150.

23. The method of claim 18, wherein the EPSs are contained in an extract.

24. The method of claim 18, wherein the EPSs are obtained by a process comprising steps of:
culturing the lactic acid bacterium to obtain a bacterial culture;
removing bacterial cell bodies from the bacterial culture to obtain a mixture containing the EPSs;
precipitating the mixture with ethanol; and
removing ethanol to obtain an extract containing the EPSs.

25. The method of claim 18, wherein the EPSs are obtained by a process comprising steps of:
culturing the lactic acid bacterium to obtain a bacterial culture;
removing bacterial cell bodies from the bacterial culture to obtain a mixture containing the EPSs;
precipitating the mixture with ethanol;
removing ethanol to obtain an extract containing the EPSs; and
obtaining a fraction having a molecular weight ranging from about 9 kDa to about 2,000 kDa from the extract.

26. The method of claim 24, wherein the extract comprises a fraction 1 having a molecular weight ranging from about 30 kDa to about 2,000 kDa, and the fraction 1 is divided by size-exclusion chromatography.

27. The method of claim 26, wherein the fraction 1 comprises a major component with a molecular weight of 1,446 kDa.

28. The method of claim 26, wherein the fraction 1 comprises fucose, galactosamine, glucosamine, galactose, glucose, and mannose.

29. The method of claim 26, wherein the fraction 1 comprises about 3.7 mol % to about 5.5 mol % fucose, about 9.0 mol % to about 13.5 mol % galactosamine, about 10.8 mol % to about 16.2 mol % glucosamine, about 27.8 mol % to about 41.7 mol % galactose, about 7.9 mol % to about 11.8 mol % glucose, and about 20.9 mol % to about 31.3 mol % mannose, wherein the total molar percentage of monosaccharide in the fraction 1 is 100 mol %.

30. The method of claim 24, wherein the extract comprises a fraction 2 having an average molecular weight ranging from about 15 kDa to about 750 kDa, and the fraction 2 is divided by size-exclusion chromatography.

31. The method of claim 24, wherein the extract comprises a fraction 3 having an average molecular weight ranging from about 9 kDa to about 380 kDa, and the fraction 3 is divided by size-exclusion chromatography.

32. The method of claim 18, wherein the pharmaceutical composition is in a form suitable for oral administration.

33. The method of claim 18, wherein the pharmaceutical composition is in the form of a solid, semi-solid, liquid, or granule of powder.

34. The method of claim 18, which is for reducing sleep latency and/or reducing recovery time from sleep.

35. The method of claim 25, wherein the extract comprises a fraction 1 having a molecular weight ranging from about 30 kDa to about 2,000 kDa, and the fraction 1 is divided by size-exclusion chromatography.

36. The method of claim 25, wherein the extract comprises a fraction 2 having an average molecular weight ranging from about 15 kDa to about 750 kDa, and the fraction 2 is divided by size-exclusion chromatography.

37. The method of claim 25, wherein the extract comprises a fraction 3 having an average molecular weight ranging from about 9 kDa to about 380 kDa, and the fraction 3 is divided by size-exclusion chromatography.
